(19) United States
(12) Patent Application Publication
(10) Pub. No.: US 2003/0194455 A1
(43) Pub. Date: Oct. 16, 2003

(54) Whole ground oriental mustard biopesticide

(75) Inventor: Thomas Dwayne Taylor, Lakeland, FL (US)

Correspondence Address: Thomas D. Taylor, 3705 Century Blvd #3, Lakeland, FL, 33811, US

(73) Assignee: Poulenger USA, Inc., Lakeland, FL

(21) Appl. No.: 10/121,345

(22) Filed: Apr. 12, 2002

(51) Int. Cl. A61K 35/78

(52) U.S. Cl. 424/755

(57) ABSTRACT

The invention provides a novel biopesticide comprised of Whole Ground Oriental Mustard for controlling soil born pathogens such as fungi and damaging nematodes. The present invention can replace many synthetically produced pesticides such as organophosphates and methyl bromide, which are damaging to the environment and to human health.

### CROSS REFERENCE TO RELATED APPLICATIONS

Publications

Brown, Paul D. & Morra, Matthew J.(1 997)“Control of Soil-Borne Plant Pest Using Glucosinolate-Containing Plants”, Advances in Agronomy, Vol 61, pp167-231.

Harvey, S. G., Hannahan, H. N. & Sams, C. E.(2001)“Indian Mustard and Allyl Isothiocyanate Inhibit Sclerotium rolfsii”, J. Amer. Soc.Hort.Sci. 127(1) pp27-31.

Borek, V., Morra, M. J., Brown, P. D., & McCaffrey, J. P.(1 995)“Transformation of the glucosinolate-derived alleochemicals allyl isothiocyanate and allyl nitrile in soil” J. Agric. Food Chem. 43, 1935-1940.

Kjaer, A. (1976)“Glucosinolates in the Cruciferae”, The Biology and Chemistry of the Cruciferae, Academic Press, pp207-219.

### BACKGROUND OF INVENTION

As environmental and human health concerns mount with the use of conventional synthetically produced pesticides, many are being banned, phased out or restricted from use. Pesticides such as organophosphates and methyl bromide, although in use now, are slowly being phased out and banned from use in the future. With the reduction in these chemicals, many end users are searching for safe environmentally friendly alternatives to these harmful products.

Biopesticides, which are pesticides that are derived from natural materials such as plants, animals, bacteria and certain minerals, has proved to be a safer alternative to conventional pesticides. The Environmental Protection Agency in 1994 set up a branch to evaluate and facilitate the registration these products under the Biopesticides & Pollution Division (BPPD) to help speed up the registration process. Since biopesticides tend to pose fewer risk than conventional pesticides, their use is encouraged.

One type of biopesticide is derived from plant materials. Certain plants have naturally occurring chemical defense mechanisms to protect itself from parasites, fungi and predators. One family of plants, the Cruciferae family, has such a defense mechanism. This family of plants contains glucosinolates and myrosinase enzymes, which upon destruction form Allyl Isothiocyanates (AITC's) and other compounds which are harmful to parasites, such as nematodes, pathogenic fungi and produces a pungent bitter taste for chewing predators.

Of the Cruciferae family, one species, brassica juncea, commonly known as Oriental or Indian Mustard, has an abundance of these compounds which would make it a prime candidate as a biopesticide in the control of root invading nematodes and pathogenic fungi.

### SUMMARY OF INVENTION

The present invention is to be utilized as a biopesticide, nematicide and fungicide in the control of damaging nematodes and pathogenic fungi in the soil. And as a replacement or alternative to synthetically produced pesticides which are harmful to the environment, humans, animals and aquatic life.

The inventor has found that naturally occurring compounds in the seed pod of the species brassica juncea, commonly known as Oriental or Indian Mustard, once ground to a power and injected into the soil as a slurry or incorporated into the soil dry, create and release compounds which are toxic to both damaging nematodes and pathogenic fungi.

The embodiment of the invention is the utilization of the whole ground harvested seedpods from the species brassica juncea. Once the seed pods are harvested the entirety of the seed pod, which include the seed with seed oils intact, the husk or hull of the seed and stem material of the seed intact, ground to a powder of varying consistencies. This powder is then incorporated into the soil either as a slurry injected via a soil injection system, or applied dry to the soil and incorporated into the soil via mechanical means.

### DETAILED DESCRIPTION

The present invention of Whole Ground Oriental Mustard Biopesticide once entering the soil by means of a soil injection system in a liquid slurry or incorporated dry into the soil by mechanical means, begins a natural degradation process in the presence of water and organic material. The glucosinolates sinigrin and gluconasturtiin contained in the Whole Ground Oriental Mustard are converted during this degradation process by the family of enzymes myrosinase to create allyl isothiocyanates (AITC's) and other biologically active products such as oxazolidinethiones (OZT's) which are released into the soil. The release of these biologically active products along with the mustard oils present in the invention, control the pathogenic fungi and suppress the damaging nematode population.

The invention may be applied in varying quantities and soil depths to produce sufficient control.

The Whole Ground Oriental Mustard Biopesticide is from the species brassica juncea and may be obtained from any variety or cultivar of that species which are commonly grown throughout the world. Once the seedpods are harvested, they remain intact until ground. The grinding process mechanically grinds the whole seed pod including the seed with oils intact, husk or hull and remaining stem material to a powder of varying consistencies to be utilized as the invention.

The Whole Ground Oriental Mustard Biopesticide as a natural organic pesticide can be handled safely and without harm to the environment or human health, as the biologically active products are formed and released once the material has entered the soil structure. Since the material is made of organic material there is associated benefits in the utilization of this invention. The invention has capabilities to retain moisture within the soil structure thereby relieving certain stresses to plants and turf caused by water infiltration problems. As an organic material, the invention contains carbohydrates and proteins which can be converted within the soil by natural degradation to food sources for plants and microbial life. The invention also adds organic matter directly to the soil structure to aid in the plants development.

Whole Ground Oriental Mustard Biopesticide may be applied with a liquid polymer, such as a commercially available product named JETWET from Poulenger USA, Inc., containing linear polymers and sulfates, to enhance the effects of the invention.

In addition a Biostimulant with high carbohydrate content, such as a commercially available product named RUTOPIA by Poulenger USA, Inc., will enhance and aid in the effects of the invention.

The following are examples of test results of the use of the invention and are for purposes of illustration and are not intended to limit the scope of the invention.

### EXAMPLE 1

The invention, Whole Ground Oriental Mustard Biopesticide, was incorporated as a slurry into the soil via the use of a soil injection system to a depth of 3 inches and at an applied rate of 150 lbs. per acre. Test plot was of a golf course green with Bermuda grass planted. The results are as follows:

Nematode assay performed and number of plant parasitic nematodes were recovered from 100 cc of soil samples.

Sampling of soil prior to treatment. (Test date 03/08/02)

| | |
|---|---|
| Sting (Belonolaimus) | 50 per 100 cc of soil |
| Lance (Hoplolaimus) | 14 per 100 cc of soil |
| Spiral (Helicotylenchus) | 55 per 100 cc of soil |
| Root Knot (Meloidogyne) | 55 per 100 cc of soil |
| Ring (Criconemella) | 792 per 100 cc of soil |
| Sheathoid (Hemicriconemodies) | 19 per 100 cc of soil |

Sampling of soil after application. Test date 03/15/02

| | |
|---|---|
| Sting (Belonolaimus) | 0 per 100 cc of soil |
| Lance (Hoplolaimus) | 0 per 100 cc of soil |
| Spiral (Helicotylenchus) | 0 per 100 cc of soil |
| Root Knot (Meloidogyne) | 0 per 100 cc of soil |
| Ring (Criconemella) | 192 per 100 cc of soil |
| Sheathoid (Hemicriconemodies) | 0 per 100 cc of soil |

### EXAMPLE 2

The invention, Whole Ground Oriental Mustard Biopesticide, was incorporated dry into the soil of 1 gallon test pots infected with strains of pathogenic fungi. The incorporation rate was equal to 150 lbs. per acre, and then water was introduced at a rate of 16 ounces per day for 7 days. The results of the test are as follows:

Treatment performed on 2/11/02, and tested for fungi on 2/18/02

| | |
|---|---|
| Pot 1: Fusarium | 100% Growth inhibition |
| Pot 2: S. rolfsii | 100% Growth inhibition |
| Pot 3: Basidiomycetes | 53% Growth inhibition |
| Pot 4: Pythium | 60% Growth inhibition |
| Pot 5: Rhizoctonia | 100% Growth inhibition |

Although the invention has been describe with detail, other versions and variations are possible. Therefore the preferred embodiments of the present invention have been described, the present invention is not limited to these preferred embodiments, but includes variations and modifications within the scope and spirit of the claims.

1. The utilization of Whole Ground brassica juncea seedpods, which include seed with oils intact, seed hull or husk and stem material ground to a powder, applied in sufficient quantities to control root invading nematodes and pathogenic fungi within the soil.

2. The method of claim 1 in the use of the seed pod of the species brassica juncea, commonly known as Oriental or Indian Mustard, containing the seed with oils intact, the seed hull or husk and stem material ground to a powder as a biopesticide, nematicide or fungicide.

3. The method of creating a slurried mixture of water and sufficient quantity of Whole Ground Oriental Mustard Biopesticide, which has been mechanically milled to a powder of varying consistencies, and injecting the mixture directly into the soil structure of established turf or plants by means of a soil injection system.
